(19) United States
(12) Patent Application Publication
(10) Pub. No.: US 2016/0271345 A1
(43) Pub. Date: Sep. 22, 2016

(54) Medicament Inhaler

(71) Applicant: NORTON (WATERFORD) LIMITED, Waterford (IE)

(72) Inventors: Julian Blair, Waterford (IE); Daniel Buck, Waterford (IE); Simon Kaar, Waterford (IE)

(73) Assignee: Norton (Waterford) Limited, Waterfrod (IE)

(21) Appl. No.: 15/033,320

(22) PCT Filed: Oct. 28, 2014
(86) PCT No.: PCT/EP2014/073141

§ 371 Date: Apr. 29, 2016

(30) Foreign Application Priority Data
Oct. 31, 2013 (GB) 1319265.3
Oct. 1, 2014 (GB) 1417412.2

(51) Int. Cl. A61M 15/00

(52) U.S. Cl. CPC A61M 15/0026; A61M 2202/064; A61M 2205/0238

(57) ABSTRACT

The invention provides a dry powder inhaler comprising a mouthpiece for patient inhalation, a cover movable about a hinge to open and close the mouthpiece, a delivery passageway for directing an inhalation induced air flow through the mouthpiece, a channel extending from the delivery passageway, a reservoir for containing medicament, the reservoir having a dispensing port connected to the channel, a cup received in the channel and movable between the dispensing port and the delivery passageway, wherein the cup comprises a cup cam follower, a cup spring biasing the cup towards the delivery passageway, a yoke movable between at least a first position and a second position and including a yoke cam, whereby closing the cover moves the yoke between the first position and the second position such that the yoke cam engages the cup cam follower and urges the cup against the cup spring to the dispensing port, and wherein the yoke cam, the cup cam follower, or both, is lubricated.

### FIELD OF THE DISCLOSURE

The present invention relates to an apparatus for administering medicament as a dry powder for inhalation by a patient and, more particularly, to a dry powder inhaler (DPI).

### BACKGROUND OF THE DISCLOSURE

DPIs are well known for dispensing medicament to the lungs of a patient, for example for treating asthma and COPD.

WO 02/00281, WO 01/097889 and WO 2005/034833 disclose an improved DPI. The DPI includes a mouthpiece for patient inhalation, a delivery passageway for directing an inhalation induced air flow through the mouthpiece, a channel extending from the delivery passageway, and a reservoir for containing medicament, with the reservoir having a dispenser port connected to the channel. The DPI also includes a cover which is pivotally mounted to the case of the inhaler for covering the mouthpiece. The inhaler has a breath-actuated mechanism: inhalation by the patient induces delivery of the medicament.

Surprisingly, following extensive investigation involving intentional extreme misuse of the inhaler, the applicant has found that increased resistance can occasionally be required to close the cover. This effect was a result of misuse involving repeated opening and closing of the inhaler cover without the medicament dose being inhaled/removed from the inhaler. In some instances the increase in force required is so great that the cover can dislocate from the inhaler body. This has significant consequences, because the closing of the cover not only protects the mouthpiece, but also resets the dosing mechanism of the DPI. Therefore, if the cover dislocates from its hinges, the DPI is no longer able to deliver medicament.

The DPI according to the present invention is adapted to address this unexpected problem arising from excessive misuse of the device.

### SUMMARY OF THE DISCLOSURE

In a first aspect the present invention provides a dry powder inhaler comprising: a mouthpiece for patient inhalation;

a cover movable about a hinge to open and close the mouthpiece;
a delivery passageway for directing an inhalation induced air flow through the mouthpiece;
a channel extending from the delivery passageway;
a reservoir for containing medicament, the reservoir having a dispensing port connected to the channel;
a cup received in the channel and movable between the dispensing port and the delivery passageway, wherein the cup comprises a cup cam follower;
a cup spring biasing the cup towards the delivery passageway;
a yoke movable between at least a first position and a second position and including a yoke cam;
whereby closing the cover moves the yoke between the first position and the second position such that yoke cam engages the cup cam follower and urges the cup against the cup spring to the dispensing port;
and wherein the yoke cam, the cam follower, or both, is lubricated.

In a second aspect the invention provides a dry powder inhaler comprising a moving component which is:
made from a plastics material which includes a slip additive; or
at least partially coated with an oil, soap or stearic acid.

The inventors have surprisingly found that the dislocation of the cover results from an increase in friction between two moving parts within the inhaler: particularly the yoke cam and the cup cam follower. The increased friction is caused by excess dry powder coating and interfering with the interaction between the components. The increased friction results in an abnormally large reset force being required to slide the cup cam follower along the yoke cam, and therefore to close the cover of the DPI. The force can be increased to the extent that the hinges of the cover become dislocated and the cup cam follower does not return the dispensing port to receive a further dose of medicament. Although the applicant does not wish to be bound by theory, it is thought that the dislocation could occur owing to a large moment being required at the hinge because of the large force applied by the yoke.

The present invention lies in the identification of this unexpected problem and the provision of its solution by reducing the friction, specifically between the cup cam follower and the yoke cam, by lubrication.

As described above, the applicant has surprisingly discovered that the problems of the increased force required to close the cover and the cover dislocating from its hinges can be solved by lubricating part of the internal surfaces of the inhaler: the yoke cam and cup cam follower.

The lubrication may be applied to reduce friction between the surfaces of the yoke cam and the cam follower which engage when the yoke cam urges the cup cam follower back to the dispensing port. Therefore, the lubricant may be applied on one or more of the surfaces of the yoke cam and the cup cam follower which engage (or are contactable in use).

The cup may be directly attached to the cup cam follower, or the cup may comprise a cup sled which guides the cup through the channel, wherein the cup cam follower is attached to the sled.

The yoke cam and the cup cam follower are typically made of a plastics material. The yoke cam can comprise a polyoxymethylene polymer. Examples of commercial products include POM Hostaform® MT8U01, MT8U03 or S9243 XAP2 and Tenac® LA541 or C LV40, POM Hostaform® MT8F01, Delrin® SC699 NC010 or Kepital® TS-25HN/25. Preferably the plastics material is a polytetrafluoroethylene-containing plastics, for example POM Hostaform® MT8F01.

The cup cam follower can comprise a polyester, for example polybutylene terephthalate. An example of a commercial product is Celanex® 2401 MT.

The surfaces of the yoke cam and/or the cup cam follower can be lubricated in a variety of ways, and suitable lubricants are known to the person skilled in the art. The term lubricated is intended to have broad meaning and include application of any lubricant or other adaptation of the surface of the components in order to reduce their coefficient of friction (which can be measured by the person skilled in the art).

In one embodiment the yoke cam is lubricated.

One embodiment of the invention involves lubrication by the application of a surface coating.

The surface coating may be a surface coating of oil, and in particular a coating of a siloxane. One particular siloxane that can be mentioned is polydimethylsiloxane. Two suitable medical grade commercial products including polydimethylsiloxane are Dow Corning 360 medical fluid and Dow Corning 365 Emulsion.

The siloxane can be applied to the component in any way. For example, by spraying the component or dipping the component in the siloxane. The component may subsequently need to be dried, for example if the siloxane is applied as an emulsion. Preferably, the oil/siloxane is applied by metering a droplet (e.g. from a syringe) directly on to the desired surface.

Alternatively, the surface coating may be a surface coating of soap or stearic acid.

A second embodiment of the invention involves lubrication by addition of a material additive.

In one example of the addition of a material additive, the yoke cam and/or the cup cam follower comprises a plastics material and the material additive in the plastics is a slip additive.

A slip additive is a plastics material modifier that acts as an internal lubricant. The slip additive is added to the plastics material during manufacture and extrudes to the surface of the plastics material during and immediately after processing thereby reducing friction and improving slip at the surface of the plastic.

The slip additive can be a siloxane. Siloxanes can be added to the plastics material by a mixing process which is compounded or as a masterbatch. An example of a siloxane masterbatch is DOW CORNING MB40006 which comprises (in wt %) 40.0-70.0% trioxane-dioxolene copolymer, 30.0-60.0% dimethylvinyl-terminated dimethyl siloxane, and 1.0-5.0% polymethylene/acetal copolymer.

Preferably the plastics material is a polytetrafluoroethylene-containing plastics material, for example POM Hostaform® MT8F01, and the slip additive is a siloxane.

Typically, the slip additive is added to the plastics material at from 1 to 10 wt %, preferably from 3-7 wt %. In one embodiment the slip additive is added at approximately 5 wt %.

In one embodiment, the DPI additionally comprises at least one cover cam mounted on the mouthpiece cover and movable with the cover between open and closed positions, wherein the cover cam includes at least a first and second cover cam surfaces;

and the yoke includes yoke cam followers which are biased against the cam surfaces by an actuation spring;
wherein the cover cam surfaces are arranged such that the yoke cam followers successively engage the first cover cam surface when the cover is closed, and the second cover cam surface when the cover is opened;
and the first cam surface is spaced further from the hinge than the second cam surfaces;
and therefore the yoke cam followers are moved by the actuation spring from the first to the second cover cam surfaces as the cover is opened and thereby the yoke is moved from the second to the first position;
and the yoke cam followers are moved against the actuation spring from the second to the first cover cam surfaces as the cover is closed and thereby the yoke is moved from the first to the second position.

Preferably the cover cam includes an additional intermediate cover cam surface between the first and second cover cam surfaces wherein the yoke cam followers are moved by the actuation spring from the first to the intermediate to the second cover cam surface as the cover is opened and the yoke cam followers are moved against the actuation spring from the second to the intermediate to the first cover cam surface as the cover is opened.

The invention also provides a dry powder inhaler comprising a moving component which is:
made from a plastics material which includes a slip additive; or
at least partially coated with an oil, soap or stearic acid.

The moving component can be a yoke cam and/or a cup cam follower as described above. Further, the moving component may be made of the materials described above, and lubricated in the manners described above.

### BRIEF DESCRIPTION OF THE DRAWINGS

Specific embodiments of the present invention will now be described with reference to the accompanying drawings, in which:

FIG. 1 shows the DPI;

FIG. 2 shows the inside of the DPI;

FIG. 3 shows the dose metering mechanism of the DPI;

FIGS. 4 and 5 show the cover of the DPI and the cover cam of the DPI;

FIGS. 6-8 show the movement of the cup to the dispensing port on closure of the cover;

FIGS. 9-11 show the movement of the cup away from the dispensing port to the delivery passageway on opening the cover;

FIG. 12 shows an unlubricated DPI for which the hinge has become dislocated;

FIG. 13 shows the movement of the cup cam follower along the yoke cam;

FIGS. 14-15 show the load required to open and close the cover of various DPIs;

FIG. 16 shows the variation of the peak closing-forces throughout the life of various inhalers; and

FIGS. 17-19 show the load required to open and close the cover of various DPIs.

### DETAILED DESCRIPTION OF THE PREFERRED EMBODIMENTS

### Description of DPI

A detailed description of the inhaler to which the lubricant is added is made in WO 02/00281, WO 01/097889 and WO 2005/034833, the contents of which are incorporated herein by reference. For a detailed description of the internal mechanisms of the inhaler, reference should be made to these earlier applications.

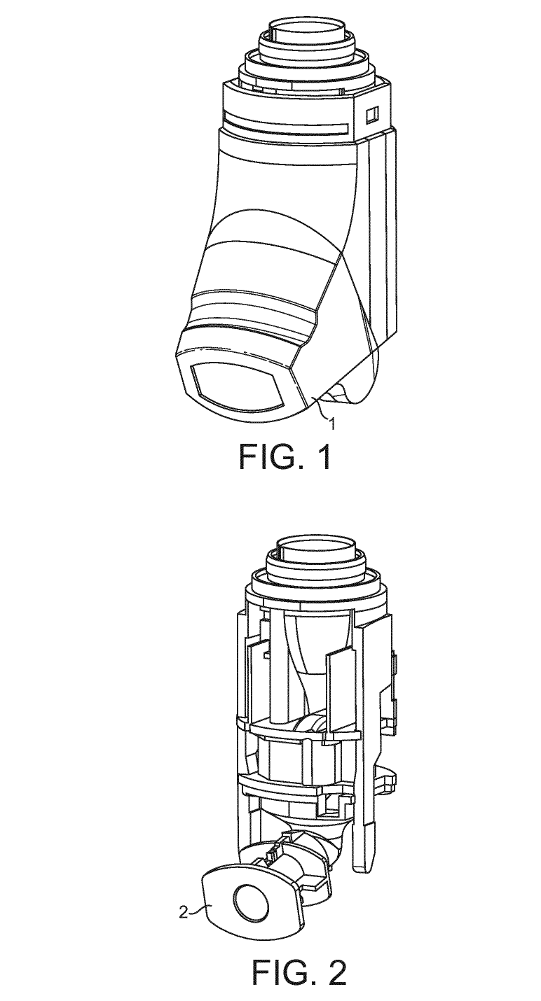

The DPI has a particular dose metering system. FIGS. 1-3 show a DPI according to the invention having a cover 1 which opens and closes about a hinge to reveal a mouthpiece 2.

The DPI has a delivery passageway for directing an inhalation induced air flow through the mouthpiece. The medicament is stored in a reservoir 3 from which it is dispensed via a dispensing port to a cup 4. The cup is movable within a channel which passes from the dispensing port to the delivery passageway. Therefore, when the cup is aligned to the dispensing port it can be filled with medicament, and when the cup moves to the delivery passageway it can be delivered to the patient, upon inhalation.

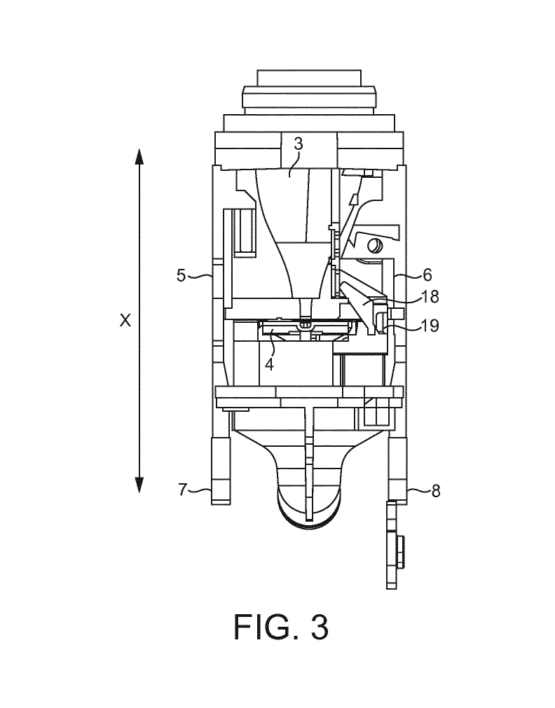

The dose metering system includes a first yoke 5 and a second yoke 6 mounted on the housing of the DPI, and movable in a linear direction parallel with an axis ‘X’ of the inhaler (see FIG. 3). An actuation spring is positioned towards the top of the DPI and biases the yokes towards the mouthpiece 2, where yoke cam followers 7,8 on the yoke engages with the cover cams on the cover 1 of the mouthpiece 2.

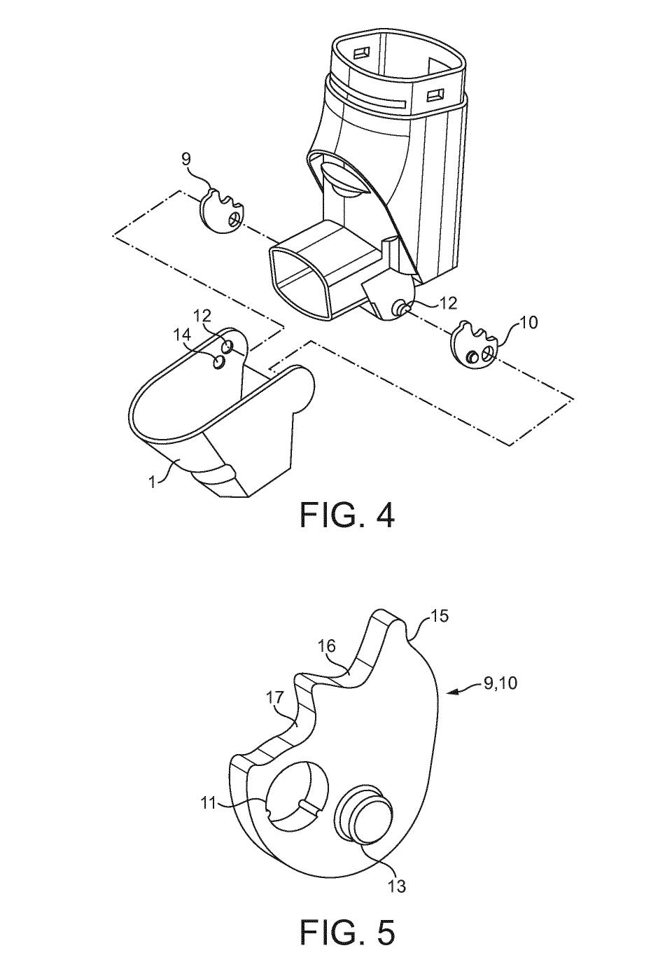

The cover of the mouthpiece is shown in more detail by FIGS. 4 and 5. Two cover cams 9,10 are mounted on the cover 1, which are movable with the cover 1 between its open and closed positions. The cams 9,10 each include an opening 11 for allowing outwardly extending hinges 12 of the case to pass therethrough and be received in first recesses 12 of the cover 1. The cover cams 9,10 also include bosses 13 extending outwardly and received in second recesses 14 of the cover 1, such that the cover 1 pivots about the hinges 12 and the cams 8,9 move with the cover 1 about the hinges 12.

Each cover cam 9,10 also includes first, intermediate and second cam surfaces 15,16,17 and the yoke cam followers 7,8 of the second yoke 6 are biased against the cam surfaces by an actuation spring. The cam surfaces 15,16,17 are arranged such the yoke cam followers 7,8 successively engage the first cam surfaces 15 when the cover 1 is closed, the intermediate cam surfaces 16 when the cover 1 is partially opened, and the second cam surfaces 17 when the cover 1 is fully opened. The first cam surfaces 15 are spaced further from the hinges 12 than the intermediate 16 and the second 17 cam surfaces, while the intermediate cam surfaces 16 are spaced further from the hinges 12 than the second cam surfaces 17. Therefore, as the cover 1 is opened the cover cams 9,10 allow the yokes 5,6 to be moved by the actuation spring parallel with the axis “X” of the inhaler in the first direction towards the mouthpiece 2. When the cover 1 is closed, the cover cams 9,10 push the yokes 5,6 parallel with the axis ‘X’ against the spring and towards the top of the inhaler.

FIG. 3 shows the cup 4 which includes a recess adapted to receive medicament from the dispenser of the reservoir 3 and sized to hold a predetermined dose of dry powdered medicament.

The cup 4 is biased to the delivery passageway by a spring attached to the DPI. As described above, when the cover 1 is closed, the yoke 5,6 moves up the inhaler. As the yoke 5,6 moves up the inhaler a yoke cam 18 attached to the yoke 6 engages a cup cam follower 19 connected to the cup 4 and urges the cup 4 against the spring to the dispensing port of the reservoir 3 where it can be filled with medicament.

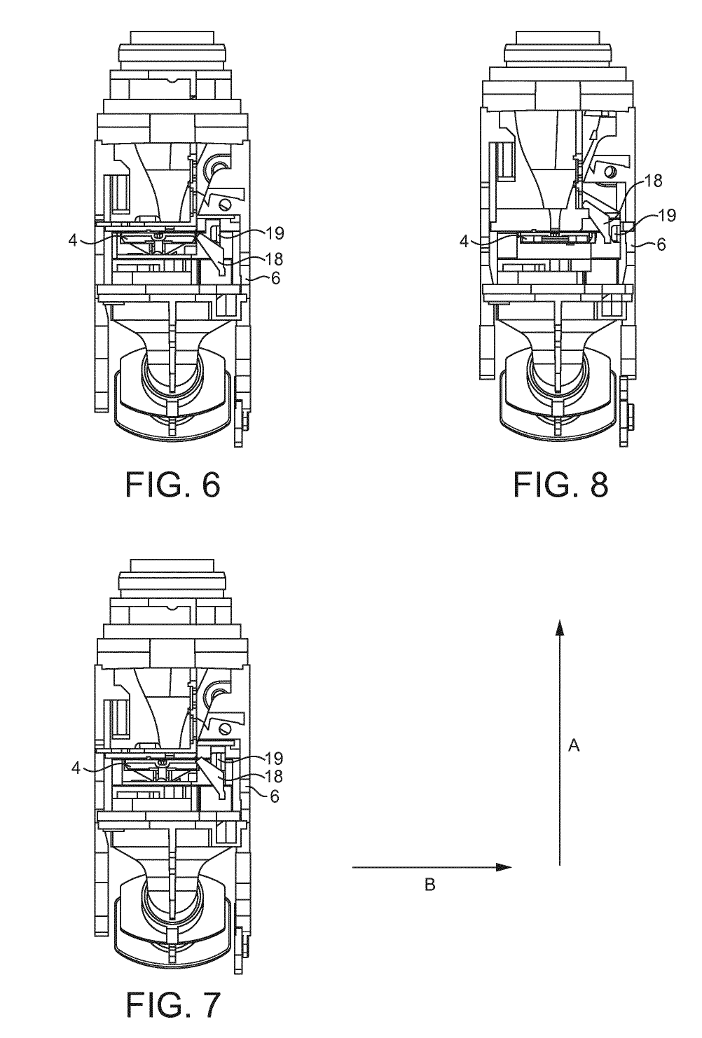

FIGS. 6 to 8 show the movement of the cup 4 from the delivery passageway to the dispensing port as the cover 1 is closed. As the cover 1 is closed, the yoke 6 moves up in direction A, and consequently the yoke cam 18 engages the cup cam follower 19, and the cam follower 19 moves along the surface of the yoke cam 18 and thus the cup 4 is moved in direction B against the cup spring, away from the delivery passageway to the dispensing port.

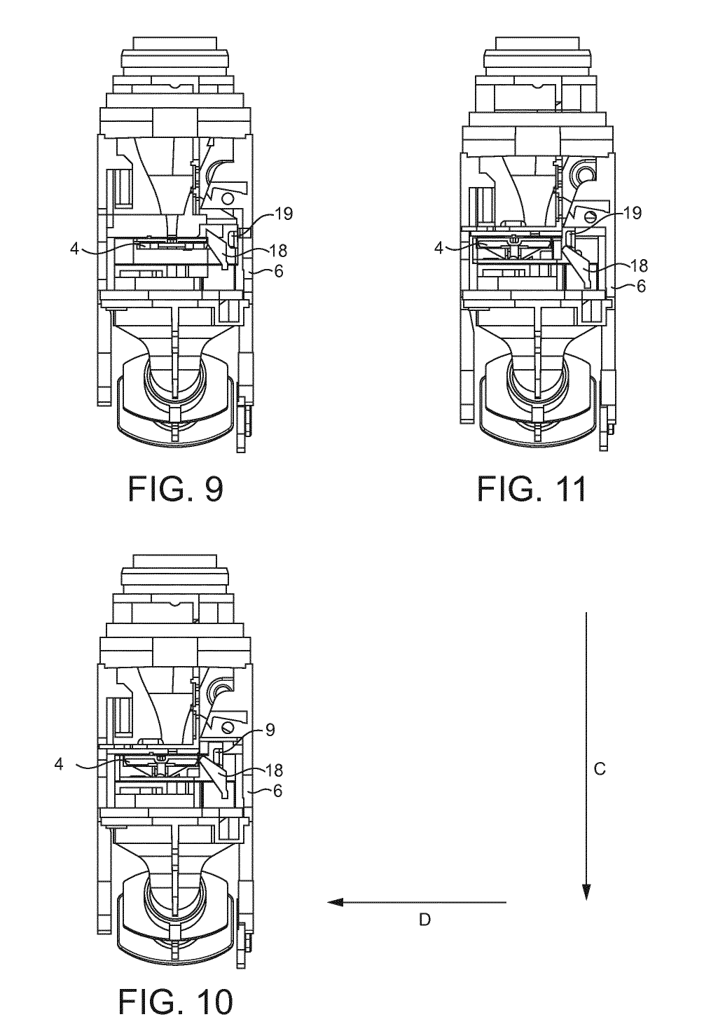

FIGS. 9 to 11 show the movement of the cup 4 from the dispensing port to the delivery passageway as the cover 1 is opened. As the yoke 6 moves down up in direction C, the yoke cam 18 releases the cup cam follower 19, and thus the cam follower 19 moves along the surface of the yoke cam 18 and the cup 4 is released in direction D by the cup spring, away from the dispensing port to the delivery passageway.

As mentioned above, the cover cam 9,10 includes an intermediate cam surface 16 upon which the yoke cam followers 7,8 engage when the cover 1 is partially open. In this position, the yoke cam 18 does not yet release the cup cam follower 19 and therefore the cup 4 remains at the dispensing port of the reservoir 3. However, in moving to the intermediate position, the yokes 5,6 partially collapse bellows in communication with the medicament reservoir 3 and thus pressurise the interior of the reservoir 3 and thereby dispense a predetermined dose of medicament to the cup 4. The specific details of the mechanism of the bellows are not material to the present invention and details can be found in WO 02/00281, WO 01/097889 and WO 2005/034833.

Since the recess of the cup 4, which holds the dose of medicament, is always either in communication with the reservoir 3 or the delivery passageway (see numeral 34 in FIG. 8 of WO 02/00281), the device can be primed and/or inhaled in any orientation, e.g. from +90° to −90° from the vertical (i.e. an upright orientation with the mouthpiece at the bottom and the reservoir at the top). Unlike many other dry powder inhalers (e.g. Turbuhaler®), the device of the present invention does not need to be used in an upright orientation.

### Medical Uses and Medicaments

The present invention is directed to inhalers for the treatment of respiratory disorders such as asthma and COPD. A range of classes of medicaments have been developed to treat respiratory disorders and each class has differing targets and effects.

Bronchodilators are employed to dilate the bronchi and bronchioles, decreasing resistance in the airways, thereby increasing the airflow to the lungs. Bronchodilators may be short-acting or long-acting. Typically, short-acting bronchodilators provide a rapid relief from acute bronchoconstriction, whereas long-acting bronchodilators help control and prevent longer-term symptoms.

Different classes of bronchodilators target different receptors in the airways. Two commonly used classes are anticholinergics and β2-agonists.

Anticholinergics (or “antimuscarinics”) block the neurotransmitter acetylcholine by selectively blocking its receptor in nerve cells. On topical application, anticholinergics act predominantly on the M3 muscarinic receptors located in the airways to produce smooth muscle relaxation, thus producing a bronchodilatory effect. Examples of long-acting muscarinic antagonists (LAMAs) include tiotropium (bromide), oxitropium (bromide), aclidinium (bromide), ipratropium (bromide) glycopyrronium (bromide), oxybutynin (hydrochloride or hydrobromide), tolterodine (tartrate), trospium (chloride), solifenacin (succinate), fesoterodine (fumarate) and darifenacin (hydrobromide). β2-Adrenergic agonists (or “β2-agonists”) act upon the β2-adrenoceptors which induces smooth muscle relaxation, resulting in dilation of the bronchial passages. Examples of long-acting β2-agonists (LABAs) include formoterol (fumarate), salmeterol (xinafoate), indacaterol (maleate), bambuterol (hydrochloride), clenbuterol (hydrochloride), olodaterol (hydrochloride), carmoterol (hydrochloride), tulobuterol (hydrochloride) and vilanterol (triphenylacetate). Examples of short-acting β2-agonists (SABAs) include albuterol

Another class of medicaments employed in the treatment of respiratory disorders are inhaled corticosteroids (ICSs). ICS are steroid hormones used in the long-term control of respiratory disorders. They function by reducing the airway inflammation. Examples include budesonide, beclomethasone (dipropionate), fluticasone (propionate), mometasone (furoate), ciclesonide and dexamethasone (sodium).

The active ingredients may be administered in combination and both combination therapies and combination products have been proposed.

Particularly preferred active ingredient for use in the present device are albuterol (sulfate), fluticasone (propionate), salmeterol (xinafoate), budesonide, formoterol (fumarate), glycopyrronium (bromide) or tiotropium (bromide). Particularly preferred fixed-dose combinations for use in the present device are fluticasone (propionate)+salmeterol (xinafoate) or budesonide+formoterol (fumarate).

### Albuterol

A preferred formulation contains racemic albuterol sulphate and lactose monohydrate. A particularly preferred formulation contains 4.7% (w/w) albuterol and 95.3% (w/w) lactose monohydrate. The albuterol may be micronized and have the following particle size distribution: d₉₀ 2.4-3.8 μm, d₅₀ 1.1-1.7 μm, d₁₀ 0.6-0.7 μm and span 1.5-2.0 μm. The lactose monohydrate is a coarse carrier and may have the following particle size distribution: d₉₀ 75-106 μm, d₅₀ 53-66 d₁₀ 19-43 μm.

The particle size distribution of the albuterol sulfate may be measured by laser diffraction as a dry dispersion, using a Sympatec HELOS/BF equipped with a RODOS disperser and ROTARY feeder. In particular, lens type R3: 0.5/0.9 . . . 175 μm is used. The following information is set on the equipment: density=3.2170 g/cm³; shape factor=1.00, calculation mode=HRLD, forced stability=0, limit curves=not used. The following trigger conditions are set: Name=Channel 28> or =2%, reference duration=10 s (single), time base=100 ms, focus prior to measurement=No, normal measurement=standard mode, start=0.000 s, Channel 28> or =2%, valid=always, stop after=5 s, channel 28< or =2%, or after=99.000 s, real time, trigger timeout=0 s repeat measurement=0 times, repeat focus=No. The following dispersion conditions are set: Name 3.0 bar, dispersing type=RODOS injector=4 mm, with=0 cascade elements, primary pressure=3.0 bar, feeder type=ROTARY, Rotation: 18%, check prim. Pres before measurement=No vacuum extraction type=Nilfisk, delay=2 s.

An adequate amount of approximately 1.0 g of the sample is weighed and filled into the groove in the rotary feeder. This is then blown by compressed air via the RODOS dry powder disperser through the measuring zone triggering a measurement. The sample particle size is measured and the D₉₀ [D(v,0.9)], D₅₀ [D(v,0.5)], D₁₀ [D(v,0,1)] and Span recorded.

The particle size distributions of the lactose may be measured by laser diffraction as a dry dispersion, using a Sympatec HELOS/BF equipped with a RODOS, RODOS/M or OASIS/M disperser and a VIBRI feeder unit. In particular, lens type R4: 0.5/4.5 . . . 350 μm is used. The following information is set on the equipment: density=1.000 g/cm³; shape factor=1.00, calculation mode=HRLD, forced stability=0. The following trigger conditions are set: Name=Optical Concentration >0.5%, reference duration=4 s (single), time base=100 ms, focus prior to measurement=yes, normal measurement=standard mode, start=0.000 s, Optical Concentration > or =0.5%, valid=0.5%< or =Channel 9< or =99.0%, stop after=1 s Optical Concentration <0.5%, or after=20.000 s, real time, trigger timeout=0 s repeat measurement=0 times, repeat focus=No. The following dispersion conditions are set: Name 1.5 bar; 75%; 1.8 mm, dispersing type=RODOS/M, injector=4 mm, with=0 cascade elements, primary pressure=1.5 bar, always auto adjust before ref. meas.=No, feeder type=VIBRI, feed rate=75%, gap width=1.8 mm, funnel rotation=0%, cleaning time=10 s, use VIBRI Control=No, vacuum extraction type=Nilfisk, delay=5 s. An adequate amount of approximately 5 g of the sample is weighed and then poured into the funnel on the VIBRI chute. This is then blown by compressed air via the RODOS dry powder disperser through the measuring zone triggering a measurement. The sample particle size is measured.

### Fluticasone Proprionate

A preferred formulation contains fluticasone proprionate and lactose monohydrate. A preferred formulation contains 3.5-4.5% (w/w) fluticasone proprionate and 95.5-96.5% (w/w) lactose monohydrate. An alternative preferred formulation contains 0.8-2.5% (w/w) fluticasone proprionate and 97.5-99.2% (w/w) lactose monohydrate. An alternative preferred formulation contains 0.4-0.6% (w/w) fluticasone proprionate and 99.4-99.6% (w/w) lactose monohydrate. The fluticasone proprionate may be micronized and have the following particle size distribution: d₉₀ 2.8-7.0 μm, d₅₀ 1.3-2.6 μm, d₁₀ 0.5-1.0 μm. The lactose monohydrate is a coarse carrier and may have the following particle size distribution: d₉₀ 140-180 μm, d₅₀ 87-107 μm, d₁₀ 30-50 μm or particle size distribution: d₉₀ 140-180 μm, d₅₀ 87-107 μm, d₁₀ 25-40 μm or size distribution: d₉₀ 140-180 μm, d₅₀ 87-107 μm, d₁₀ 17-32 μm or size distribution: d₉₀ 140-180 μm, d₅₀ 87-107 μm, d₁₀ 10-25 μm.

### Fluticasone Proprionate

The particle size of the fluticasone propionate may be measured by laser diffraction as an aqueous dispersion, e.g. using a Malvern Mastersizer 2000 instrument. In particular, the technique is wet dispersion. The equipment is set with the following optical parameters: Refractive index for fluticasone propionate=1.530, Refractive index for dispersant water=1.330, Absorption=3.0 and Obscuration=10-30%. The sample suspension is prepared by mixing approximately 50 mg sample with 10 ml of de-ionized water containing 1% Tween 80 in a 25 ml glass vessel. The suspension is stirred with a magnetic stirrer for 2 min at moderate speed. The Hydro 2000 S dispersion unit tank is filled with about 150 ml de-ionized water. The de-ionized water is sonicated by setting the ultrasonics at the level of 100% for 30 seconds and then the ultrasonic is turned back down to 0%. The pump/stirrer in the dispersion unit tank is turned to 3500 rpm and then down to zero to clear any bubbles. About 0.3 ml of 1% TA-10X FG defoamer is added into the dispersion media and the pump/stirrer is turned to about 2000 rpm and then the background is measured. The prepared suspension samples are slowly dropped into the dispersion unit until a stabilized initial obscuration at 10-20% is reached. The sample is continued to be stirred in the dispersion unit for about 1 min at 2000 rpm, then the ultrasound is turned on and the level set to 100%. After sonicating for 5 min with both the pump and ultrasound on, the sample is measured three times. The procedure is repeated two more times.

### Lactose

The particle size distribution of the lactose provided herein may be measured by laser diffraction as a dry dispersion in air, e.g. with a Sympatec HELOS/BF equipped with a RODOS disperser and a VIBRI feeder unit. In particular, lens type R5: 0.5/4.5 . . . 875 μm is used. The following information is set on the equipment: density=1.5500 g/cm³; shape factor=1.00, calculation mode=HRLD, forced stability=0. The following trigger conditions are set: Name=CH12, 0.2%, reference duration=10 s (single), time base=100 ms, focus prior to measurement=yes, normal measurement=standard mode, start=0.000 s, channel 12≧0.2%, valid=always, stop after=5.000 s, channel 12≦0.2%, or after=60.000 s, real time, repeat measurement=0, repeat focus=No. The following dispersion conditions are set: Name 1.5 bar; 85%; 2.5 mm, dispersing type=RODOS/M, injector=4 mm, with=0 cascade elements, primary pressure=1.5 bar, always auto adjust before ref. meas.=No, feeder type=VIBRI, feed rate=85%, gap width=2.5 mm, funnel rotation=0%, cleaning time=10 s, use VIBRI Control=No, vacuum extraction type=Nilfisk, delay=5 s. An adequate amount of approximately 5 g of the sample is transferred into a weighing paper using a clean dry stainless steel spatula and then poured into the funnel on the VIBRI chute. The sample is measured. The pressure is maintained at about 1.4-1.6 bar, measurement time=1.0-10.0 seconds, Cₒₚₜ=5-15% and vacuum < or =7 mbar. The procedure is repeated two more times.

### Budesonide+Formoterol Combination

A preferred formulation contains budesonide, formoterol fumarate dihydrate and lactose monohydrate. The budesonide may be micronized and have the following particle size distribution: d₉₀<10 μm, d₅₀<5 μm, d₁₀<1 μm and NLT 99%<10 μm, preferably the budesonide may have the following particle size distribution: d₉₀ 2.5-8.0 μm, d₅₀ 1-5 μm, d₁₀ 0.5-1.5 μm and NLT 99%<10 μm. More preferably the budesonide may have the following particle size distribution: d₉₀ 3-6 μm, d₅₀ 1-3 μm, d₁₀<1 μm and NLT 99%<10 μm. The delivered dose of budesonide is preferably 50-500 μg per actuation, with specific examples being 80, 160 and 320 μg per actuation.

The formoterol may be micronized and have the following particle size distribution: d₉₀<10 μm, d₅₀<5 μm, d₁₀<1 μm, NLT 99%<10 μm, preferably the formoterol may have the following particle size distribution: d₉₀ 2.5-8.0 μm, d₅₀ 1-5 μm, d₁₀ 0.5-15 μm and NLT 99%<10 μm, more preferably the formoterol may have the following particle size distribution: d90 3.5-6.0 μm, d50 1-3 μm, d₁₀<1 μm and NLT 99%<10 μm. The delivered dose of formoterol fumarate, as base, is preferably 1-20 μg per actuation, with specific examples being 4.5 and 9 μg per actuation. The doses are based on the amount of formoterol present (i.e. the amount is calculated without including contribution to the mass of the counter ion).

Particularly preferred delivered doses of budesonide/formoterol in μg are 80/4.5, 160/4.5 and 320/9.

The lactose monohydrate is a coarse carrier and may have the following particle size distribution: d₉₀ 130-180 μm, d₅₀ 80-120 μm, d₁₀ 20-65 μm and <10 μm=<10%, preferably <10 μm=<6%, preferably the lactose may have the following particle size distribution: d₉₀ 130-180 μm, d₅₀ 80-120 μm, d₁₀ 10-60 μm and NMT 99%<10 μm.

### Budesonide

The particle size distributions of budesonide may be measured by laser diffraction as a dry dispersion, e.g. in air, such as in a Sympatec HELOS/BF equipped with a RODOS disperser and an ASPIROS feeder unit. In particular, lens type R1: 0.1/0.18 . . . 35 μm is used. The following information is set on the equipment: density=1.000 g/cm³; shape factor=1.00, calculation mode=HRLD, forced stability=0 and limited curves=not used. The following trigger conditions are set: Name=CH25>0.5%_50 ms, reference duration=10 s (single), time base=50 ms, focus prior to measurement=yes, mode=fast mode, start series=0.000 s, channel 25≧0.5%, valid=always, stop series after=0.500 s real time, trigger time out 10 s and split series of =500 ms real time. The following dispersion conditions are set: Name 3.5 bar 60 mm/s, dispersing type=RODOS/M, injector=4 mm, with=0 cascade elements, primary pressure=3.5 bar, always auto adjust before ref. meas.=No, feeder type=ASPIROS, speed=60 mm/s, vacuum extraction type=Nilfisk, delay=5 s. An adequate amount of approximately 20 mg of the sample is transferred into an ASPIROS tube using a clean dry stainless steel spatula and then installed into the APIROS feeder. The sample is measured. The pressure is maintained at about 3.4-3.6 bar, Cₒₚₜ=6.0-12% and vacuum ≦70 mbar.

### Formoterol

The particle size distributions of formoterol may be measured by laser diffraction as a dry dispersion, e.g. in air, such as in a Sympatec HELOS/BF equipped with a RODOS disperser and an ASPIROS feeder unit. In particular, lens type R1: 0.1/0.18 . . . 35 μm is used. The following information is set on the equipment: density=1.000 g/cm³; shape factor=1.00, calculation mode=HRLD, forced stability=0 and limited curves=not used. The following trigger conditions are set: Name=CH21>0.5%_100 ms, reference duration=10 s (single), time base=100 ms, focus prior to measurement=yes, mode=standard mode, start series=0.000 s, channel 21≧0.5%, valid=always, stop series after=0.500 s real time, trigger time out 10 s, repeat measurement=0 times and repeat focus=no. The following dispersion conditions are set: Name 3.5 bar 60 mm/s, dispersing type=RODOS/M, injector=4 mm, with=0 cascade elements, primary pressure=3.5 bar, always auto adjust before ref. meas.=No, feeder type=ASPIROS, speed=60 mm/s, vacuum extraction type=Nilfisk, delay=5 s. An adequate amount of approximately 20 mg of the sample is transferred into an ASPIROS tube using a clean dry stainless steel spatula and then installed into the APIROS feeder. The sample is measured. The pressure is maintained at about 3.4-3.6 bar, Copt=6.0-12% and vacuum ≦70 mbar.

### Lactose

The particle size distributions of the lactose may be measured by laser diffraction as a dry dispersion, e.g. in air, such as in a Sympatec HELOS/BF equipped with a RODOS disperser and a VIBRI feeder unit. In particular, lens type R5: 0.5/4.5 . . . 875 μm is used. The following information is set on the equipment: density=1.5500 g/cm³; shape factor=1.00, calculation mode=HRLD, forced stability=0 and limited curves=not used. The following trigger conditions are set: Name=CH12, 0.2%, reference duration=10 s (single), time base=100 ms, focus prior to measurement=yes, normal measurement=standard mode, start=0.000 s, channel 12≧0.2%, valid=always, stop after=5.000 s, channel 12≦0.2%, or after=60.000 s, real time, repeat measurement=0, repeat focus=No. The following dispersion conditions are set: Name 1.5 bar; 85%; 2.5 mm, dispersing type=RODOS/M, injector=4 mm, with=0 cascade elements, primary pressure=1.5 bar, always auto adjust before ref. meas.=No, feeder type=VIBRI, feed rate=85%, gap width=2.5 mm, funnel rotation=0%, cleaning time=10 s, use VIBRI Control=No, vacuum extraction type=Nilfisk, delay=5 s. An adequate amount of approximately 5 g of the sample is transferred into the funnel on the VIBRI using a clean dry stainless steel spatula. The sample is measured. The pressure is maintained at about 1.4-1.6 bar, measurement time=1.0-10.0 seconds, Copt=5-15% and vacuum ≦70 mbar.

### Fluticasone+Salmeterol Combination

A preferred formulation may contain fluticasone proprionate, salmeterol xinafoate and lactose monohydrate. A preferred formulation contains 1% (w/w) fluticasone proprionate, 0.5-1.0% (w/w) salmeterol xinafoate and lactose monohydrate. A preferred formulation contains 2.5% (w/w) fluticasone proprionate, 0.5-1.0% (w/w) salmeterol xinafoate and lactose monohydrate. A preferred formulation contains 5% (w/w) fluticasone proprionate, 0.5-1.0% (w/w) salmeterol xinafoate and lactose monohydrate.

The fluticasone proprionate may be micronized and have the following particle size distribution: d₁₀ 0.4-1.1 μm, d₅₀ 1.1-3.0 μm, d₉₀ 2.6-7.5 μm and NLT 95%<10 μm. The fluticasone proprionate may be micronized and have the following particle size distribution: d₁₀ 0.5-1.0 μm, d₅₀ 1.8-2.6 μm, d₉₀ 3.0-6.5 μm and NLT 99%<10 μm. The fluticasone proprionate may be micronized and have the following particle size distribution: d₁₀ 0.5-1.0 μm, d₅₀ 1.90-2.50 μm, d₉₀ 3.5-6.5 μm and NLT 99%<10 μm.

The particle size (d₅₀) of fluticasone proprionate may be 1.4-2.4 μm.

The salmeterol xinafoate may be micronized and have the following particle size distribution: d₁₀ 0.4-1.3 μm, d₅₀ 1.4-3.0 μm, d₉₀ 2.4-6.5 μm and NLT 95%<10 μm. The salmeterol xinafoate may be micronized and have the following particle size distribution: d₁₀ 0.6-1.1 μm, d₅₀ 1.75-2.65 μm, d₉₀ 2.7-5.5 μm and NLT 99%<10 μm. The fluticasone proprionate may be micronized and have the following particle size distribution: d₁₀ 0.7-1.0 μm, d₅₀ 2.0-2.4 μm, d₉₀ 3.9-5.0 μm and NLT 99%<10 μm.

The particle size (d₅₀) of salmeterol xinafoate may be 1.4-2.4 μm.

It is preferable that substantially all of the particles of lactose are less than 300 μm in size. It is preferable that the lactose carrier includes a portion of fine material, that is, lactose particles of less than 10 μm in size. The fine lactose may be present in an amount of 1-10 wt %, more preferably 2.5-7.5 wt %, based on the amount of lactose. Preferably the particle size distribution of the lactose fraction is d₁₀=15-50 μm, d₅₀=80-120 μm, d₉₀=120-200 μm, NLT 99%<300 μm and 1.5-8.5%<10 μm. Preferably the particle size distribution of the lactose fraction is d₁₀=25-40 μm, d₅₀=87-107 d₉₀=140-180 μm, NLT 99%<300 μm and 2.5-7.5%<10 μm.

The lactose may include non-micronised lactose coarse lactose: (particle size (d₅₀) of 100-250 μm, by sieving) and fine lactose (particle size (d₅₀) of 1.3-3.5 μm, by jet micronisation).

### Fluticasone Proprionate

The particle size of the fluticasone propionate may be measured by laser diffraction as an aqueous dispersion, e.g. using a Malvern Mastersizer 2000 instrument. In particular, the technique is wet dispersion. The equipment is set with the following optical parameters: Refractive index for fluticasone propionate=1.530, Refractive index for dispersant water=1.330, Absorption=3.0 and Obscuration=10-30%. The sample suspension is prepared by mixing approximately 50 mg sample with 10 ml of de-ionized water containing 1% Tween 80 in a 25 ml glass vessel. The suspension is stirred with a magnetic stirrer for 2 min at moderate speed. The Hydro 2000 S dispersion unit tank is filled with about 150 ml de-ionized water. The de-ionized water is sonicated by setting the ultrasonics at the level of 100% for 30 seconds and then the ultrasonic is turned back down to 0%. The pump/stirrer in the dispersion unit tank is turned to 3500 rpm and then down to zero to clear any bubbles. About 0.3 ml of 1% TA-10X FG defoamer is added into the dispersion media and the pump/stirrer is turned to about 2000 rpm and then the background is measured. The prepared suspension samples are slowly dropped into the dispersion unit until a stabilized initial obscuration at 10-20% is reached. The sample is continued to be stirred in the dispersion unit for about 1 min at 2000 rpm, then the ultrasound is turned on and the level set to 100%. After sonicating for 5 min with both the pump and ultrasound on, the sample is measured three times. The procedure is repeated two more times.

### Salmeterol Xinafoate

The particle size of the salmeterol xinafoate may be measured using the same methodology as described for fluticasone proprionate. In particular, the technique is wet dispersion. The equipment is set with the following optical parameters: Refractive index for salmeterol xinafoate=1.500, Refractive index for dispersant water=1.330, Absorption=0.1 and Obscuration=10-30%. The sample suspension is prepared by mixing approximately 50 mg sample with 10 ml of de-ionized water containing 1% Tween 80 in a 25 ml glass vessel. The suspension is stirred with a magnetic stirrer for 2 min at moderate speed. The Hydro 2000 S dispersion unit tank is filled with about 150 ml de-ionized water. The de-ionized water is sonicated by setting the ultrasonics at the level of 100% for 30 seconds and the ultrasonic is turned back down to 0%. The pump/stirrer in the dispersion unit tank is turned to 3500 rpm and then down to zero to clear any bubbles. About 0.3 ml of 1% TA-10X FG defoamer is added into the dispersion media and the pump/stirrer is turned to about 2250 rpm and then the background is measured. The prepared suspension samples are slowly dropped into the dispersion until a stabilized initial obscuration at 15-20% is reached. The sample is continued to be stirred in the dispersion unit for about 1 min at 2250 rpm, then the ultrasound is turned on and the level set to 100%. After sonicating for 3 min with both the pump and ultrasound on, the sample is measured three times. The procedure is repeated two more times.

### Lactose

The particle size distribution of the lactose provided herein may be measured by laser diffraction as a dry dispersion in air, e.g. with a Sympatec HELOS/BF equipped with a RODOS disperser and a VIBRI feeder unit. In particular, lens type R5: 0.5/4.5 . . . 875 m is used. The following information is set on the equipment: density=1.5500 g/cm3; shape factor=1.00, calculation mode=HRLD, forced stability=0. The following trigger conditions are set: Name=CH12, 0.2%, reference duration=10 s (single), time base=100 ms, focus prior to measurement=yes, normal measurement=standard mode, start=0.000 s, channel 12≧0.2%, valid=always, stop after=5.000 s, channel 12≦0.2%, or after=60.000 s, real time, repeat measurement=0, repeat focus=No. The following dispersion conditions are set: Name 1.5 bar; 85%; 2.5 mm, dispersing type=RODOS/M, injector=4 mm, with=0 cascade elements, primary pressure=1.5 bar, always auto adjust before ref. meas.=No, feeder type=VIBRI, feed rate=85%, gap width=2.5 mm, funnel rotation=0%, cleaning time=10 s, use VIBRI Control=No, vacuum extraction type=Nilfisk, delay=5 s. An adequate amount of approximately 5 g of the sample is transferred into a weighing paper using a clean dry stainless steel spatula and then poured into the funnel on the VIBRI chute. The sample is measured. The pressure is maintained at about 1.4-1.6 bar, measurement time=1.0-10.0 seconds, Copt=5-15% and vacuum < or =7 mbar. The procedure is repeated two more times.

The active ingredients are administered by inhalation for the treatment of respiratory disorders. A number of approaches have been taken in formulating these active ingredients for delivery by inhalation, such as via a dry powder inhaler (DPI), a pressurised metered dose inhaler (pMDI) or a nebuliser.

Generally, powdered medicament particles suitable for delivery to the bronchial or alveolar region of the lung have an aerodynamic diameter of less than 10 μm, preferably less than 6 μm. Other sized particles may be used if delivery to other portions of the respiratory tract is desired, such as the nasal cavity, mouth or throat. The medicament may be delivered as pure drug, but more appropriately, it is preferred that medicaments are delivered together with excipients (carriers) which are suitable for inhalation. Suitable excipients include organic excipients such as polysaccharides (e.g. starch, cellulose and the like), lactose, glucose, mannitol, amino acids, and maltodextrins, and inorganic excipients such as calcium carbonate or sodium chloride. Lactose is a preferred excipient.

Particles of powdered medicament and/or excipient may be produced by conventional techniques, for example by micronisation, milling or sieving.

Additionally, medicament and/or excipient powders may be engineered with particular densities, size ranges, or characteristics. Particles may comprise active agents, surfactants, wall forming materials, or other components considered desirable by those of ordinary skill.

### Misuse of Non-Lubricated Inhaler

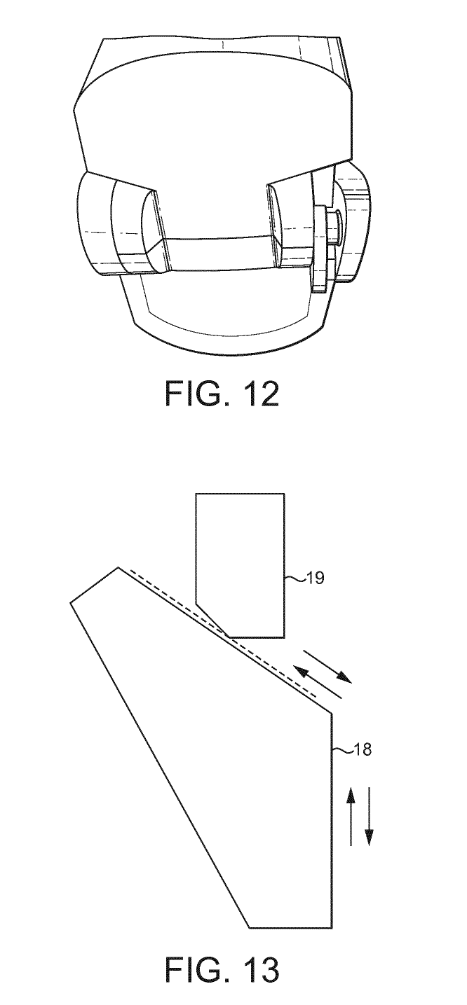

It was found that following misuse of a non-lubricated DPI (not according to the invention) by repeatedly opening and closing the cover without removing the dispensed dose, a proportion of covers dislocated from the inhaler. An example of an inhaler for which the cover has become dislocated is shown by FIG. 12. It was found that the peak force required for the inhalers which failed was far greater than for the inhalers which did not fail. The peak force is the maximum force required during closing of the inhaler cover.

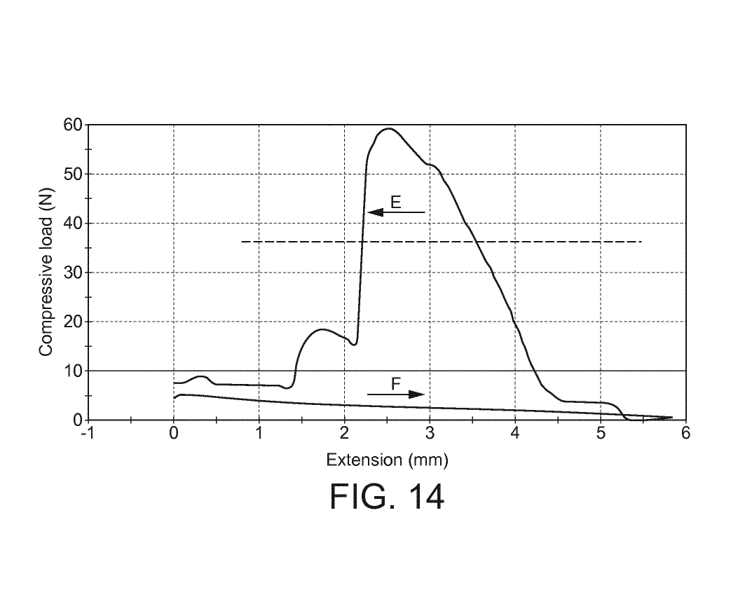

The forces required to open and close the cover of the DPI were measured for a non-lubricated inhaler. The results are shown in FIG. 14. The load-extension required to open the cover of the inhaler is shown by F, and the load-extension required to close the inhaler is shown by E. It can be seen that the maximum load required is on closing the inhaler.

Experiments with Lubricated Inhalers

### (i) Lubrication by Application of Silicone Coating

A yoke cam of a DPI according to the invention was lubricated using the following methods:

(a) spraying with silicone;
(b) brushing with Dow Corning 360 Medical Fluid Devices; and
(c) applying a 5 μl droplet of Dow Corning 360 Medical Fluid.

### Results

(a) The cover was opened and closed up to around 320 times without failure. The mean/peak closing forces was 23/31 N. The experiment was repeated ten times.
(b) The cover was opened and closed up to around 200 times without failure. The mean/peak closing force was 20/27N. The experiment was repeated ten times.
(c) The cover was opened and closed up to around 200 times without failure. The mean/peak closing force was 36/26N. The experiment was repeated four times.

It is envisaged that on a commercial scale the yokes could be coated either by immersion in a silioxane emulsion and then drying, or by spraying tumbling yokes with a silioxane emulsion.

### (ii) Lubrication by Adding an Anti-Slip Additive

A yoke according to the invention comprising a plastics material including an anti-slip additive can be prepared by addition of siloxane whilst injection molding the yoke.

Yokes were prepared comprising polyoxymethylene-dioxolane copolymer, with either 0%, 1%, 3%, or 5% of a siloxane anti-slip additive.

The inhalers were intentionally mistreated by which we mean the absence of inhaling between shots during application of 200 shots without air flow. The experiment was repeated 4 times for each proportion of siloxane anti-slip additive (table 1).

TABLE 1

| Siloxane | Failure rate |
|---|---|
| 0% | 100% |
| 1% | 75% |
| 3% | 75% |
| 5% | 25% |

The metrology of the composition were within specification at all additive rates, and mechanical testing confirmed the hardness of the plastics material was the same.

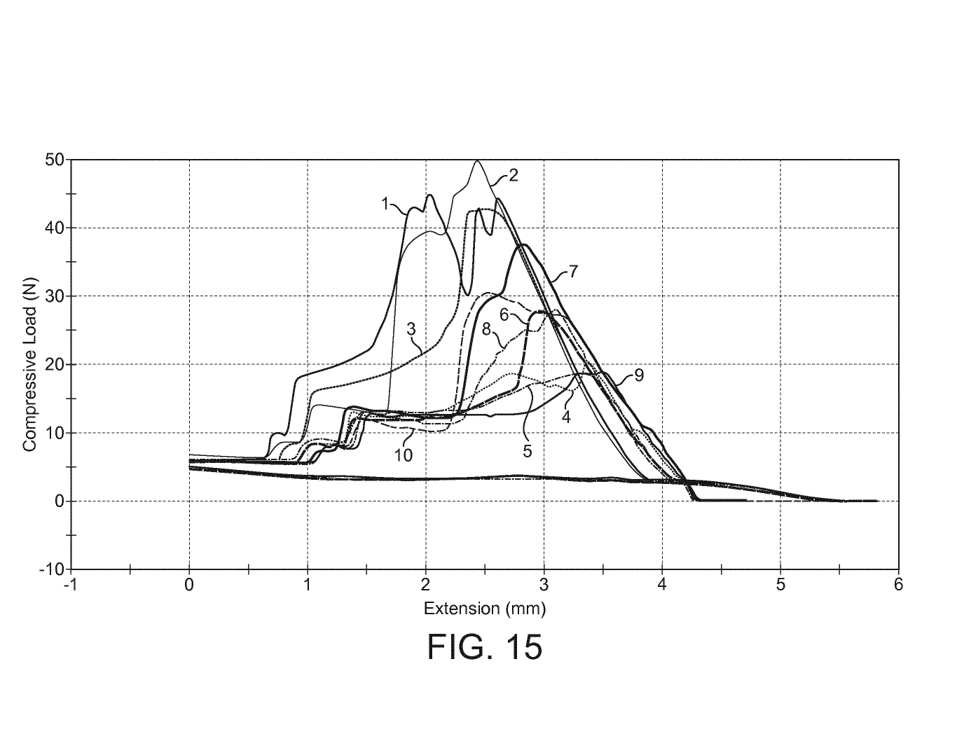

The load required to open and close the covers of the inhalers was also measured. The results of ten inhalers which were either control (included 0% siloxane slip additive) or contained 5% siloxane slip additive are illustrated by FIG. 15.

It can be seen that the maximum loads (shown in Table 2) are lower for the yokes which contain 5% siloxane slip additive than the controls which contain 0% slip additive.

TABLE 2

| | Inhaler | Maximum compressive |
|---|---|---|
| Description | label | load (N) |
| Control | 1 | 45.088 |
| | 2 | 49.742 |
| | 3 | 42.604 |
| 5% siloxane | 4 | 20.195 |
| | 5 | 18.652 |
| | 6 | 27.898 |
| | 7 | 37.662 |
| | 8 | 28.019 |
| | 9 | 18.337 |
| | 10 | 30.449 |
| Max control | | 49.742 |
| Max 5% siloxane | | 37.662 |
| Mean control | | 45.811 |
| Mean 5% siloxane | | 29.959 |

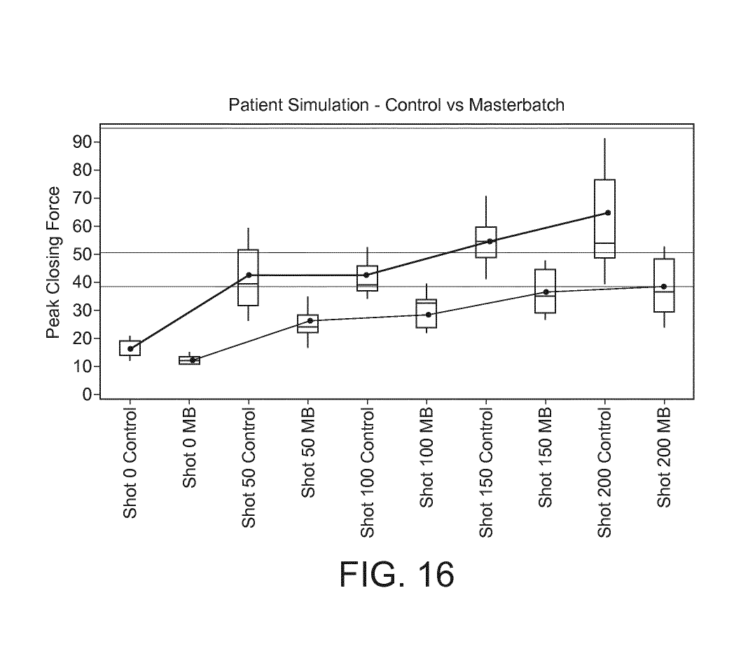

FIG. 16 shows a comparison of the maximum closing forces following simulated misuse for inhalers with yoke cams comprising POM Hostaform MT8F01 and either 0% siloxane slip additive (upper line) or contained 5% siloxane slip additive (lower line). The simulated misuse was as follows (table 3):

TABLE 3

| Shot number | Actuation description |
|---|---|
| 1-5 | Open in vertical orientation, inhale at 65 litres |
| | per minute (LPM), close vertically |
| 6-10 | Open in horizontal orientation, inhaler at 65 LPM, |
| | close vertically |
| 11-15 | Open horizontally, block inlet, inhale at 65 LPM, |
| | close horizontally |
| 16-20 | Open inverted, inhale at 20 LP, close inverted |
| 21-25 | Open vertically, no inhale, close vertically |
| 26-30 | Open in vertical orientation, inhale at 65 LPM, |
| | close vertically |
| 31-35 | Open in horizontal orientation, inhale at 65 LPM, |
| | close vertically |
| 36-40 | Open horizontally, block inlet, inhale at 65 LPM, |
| | close horizontally |
| 41-45 | Open inverted, inhale at 20 LPM, close inverted |
| 46-50 | Open vertically, no inhale, close vertically |

The misuse described in Table 3 was repeated four times, making 200 shots in total.

FIG. 16 shows that the peak closing-force is higher for the unlubricated inhaler (upper line). A force of greater than 50N results in the risk of dislocation.

(iii) Lubrication by Soap

Yoke cams according to the invention were lubricated using:

(i) commercially available soap 1 (inhalers 1 and 2); or
(ii) commercially available soap 2 (inhalers 3 and 4); or
(iii) talc (inhalers 5 and 6).

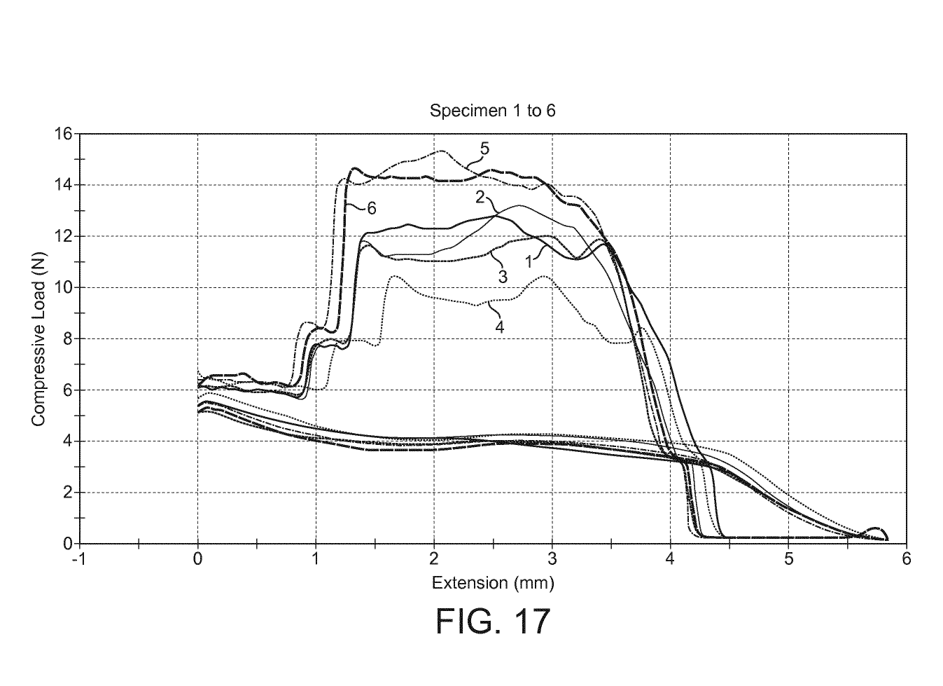

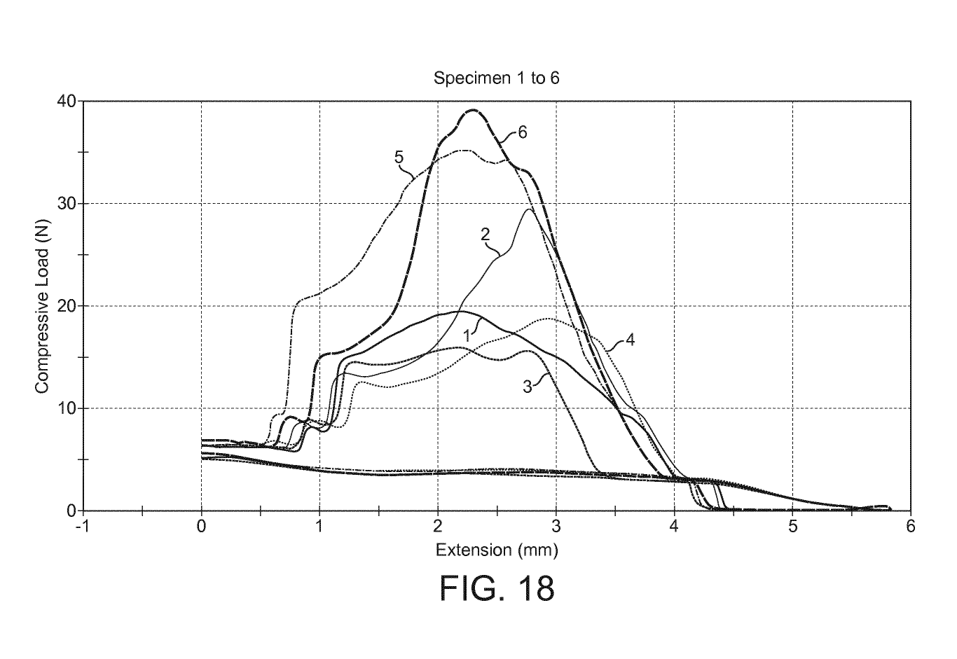

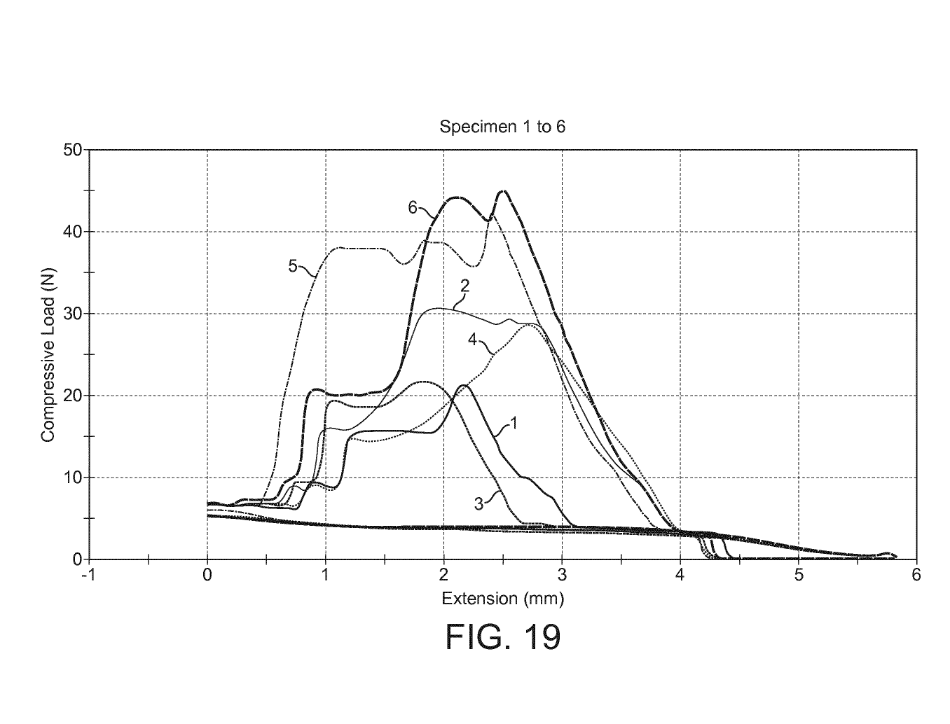

The inhalers were actuated for 200 shots without airflow. The load required to open and close the covers of the inhalers was measured at the start (FIG. 17; table 4), middle (FIG. 18; table 5) and end (FIG. 19; table 6) of the process i.e. after 0 actuations, about 100 actuations and after 200 actuations.

TABLE 4

| | Maximum compressive |
|---|---|
| Inhaler | load (N) |
| 1 | 12.794 |
| 2 | 13.230 |
| 3 | 12.020 |
| 4 | 10.464 |
| 5 | 15.386 |
| 6 | 14.659 |
| Maximum | 15.386 |
| Minimum | 10.464 |
| Mean | 13.092 |
| Standard Deviation | 1.782 |

TABLE 5

| | Maximum compressive |
|---|---|
| Inhaler | load (N) |
| 1 | 19.399 |
| 2 | 29.592 |
| 3 | 15.956 |
| 4 | 18.774 |
| 5 | 35.188 |
| 6 | 39.102 |
| Maximum | 39.102 |
| Minimum | 15.956 |
| Mean | 26.335 |
| Standard Deviation | 9.643 |

TABLE 6

| | Maximum compressive |
|---|---|
| Inhaler | load (N) |
| 1 | 21.497 |
| 2 | 30.575 |
| 3 | 21.600 |
| 4 | 28.663 |
| 5 | 42.534 |
| 6 | 45.088 |
| Maximum | 45.088 |
| Minimum | 21.497 |
| Mean | 31.659 |
| Standard Deviation | 10.131 |

It can be seen that both soaps performed better than talc, because the maximum forces required were lower.

### Orientation Study

A Spiromax® (Teva Pharmaceuticals Ltd) multi-dose dry powder inhaler was charged with a formulation containing albuterol sulfate (4.7% w/w) and alpha-lactose monohydrate (95.3% w/w), providing an emitted dose of 90 μg albuterol base per actuation.

Delivered-dose uniformity (DDU) testing was carried out on three devices at metering orientations of −90° (supine, position, mouthpiece down), −45°, 0° (perfectly upright), +45° and +90° (prone position, mouthpiece up) and dose-removal orientations of 0° (perfectly upright) and −90° (supine position). The devices were assessed at the beginning (actuation 1), middle (actuation 99) and end (actuation 200) of device life.

Aerodynamic particle size distribution (APSD) testing was carried out on three devices from each batch at metering orientations of −90°, (supine, position, mouthpiece down), −45°, 0° (perfectly upright), +45° and +90° (prone position, mouthpiece up) and dose-removal orientations of 0° (perfectly upright) and −90° (supine position). The devices were assessed at the beginning (actuations 21-30) and at the end (actuations 171-180) of device life.

DDU and APSD results are set out in Tables 7-9. Table 7 sets out a summary of the DDU results for three albuterol MDPI, labelled batches “AB1001”, “AB1002” and “AB1004” at dose removal orientations of 0° and −90°. Tables 9 and 10 set out a summary of the APSD results for three albuterol MDPI, labelled batches “AB1001”, “AB1002” and “AB1004” at dose removal orientations of 0° and −90°, respectively.

TABLE 7

| Batch | Test | Dose Metering Orientation | | | | |
|---|---|---|---|---|---|---|
| Number | Parameter | −90° | −45° | 0° | +45° | +90° |

Dose Removal Orientation at 0° (% LCED)

| | | | | | | |
|---|---|---|---|---|---|---|
| AB1001 | Mean | 96 | 97 | 98 | 94 | 97 |
| (n = 9) | Range | 90-102 | 91-104 | 89-109 | 87-101 | 88-104 |
| AB1002 | Mean | 102 | 95 | 98 | 97 | 100 |
| (n = 9) | Range | 93-114 | 89-98 | 93-105 | 88-104 | 93-114 |
| AB1004 | Mean | 95 | 99 | 101 | 93 | 95 |
| (n = 9) | Range | 87-99 | 93-105 | 91-105 | 87-99 | 90-101 |

Dose Removal Orientation at −90° (% LCED)

| | | | | | | |
|---|---|---|---|---|---|---|
| AB1001 | Mean | 100 | 93 | 97 | 97 | 100 |
| (n = 9) | Range | 88-109 | 86-99 | 91-103 | 91-104 | 90-122 |
| AB1002 | Mean | 97 | 95 | 94 | 98 | 97 |
| (n = 9) | Range | 89-109 | 90-102 | 91-98 | 88-108 | 88-106 |
| AB1004 | Mean | 99 | 99 | 96 | 98 | 100 |
| (n = 9) | Range | 85-117 | 94-102 | 89-102 | 94-106 | 90-115 |

TABLE 8

| Metering | Test | Group 1 | Group 2 | Group 3 | Group 4 | Mass Balance |
|---|---|---|---|---|---|---|
| Orientation | Parameter | (36-58 mcg) | (1-3 mcg) | (22-44 mcg) | (4-17 mcg) | (85-115%) |

AB1001

| | | | | | | |
|---|---|---|---|---|---|---|
| −90° | Mean (mcg) | 43 | 2 | 34 | 10 | 98 |
| (n = 6) | Range (mcg) | 41-45 | 2-3 | 32-35 | 8-11 | 96-104 |
| −45° | Mean (mcg) | 42 | 2 | 34 | 9 | 96 |
| (n = 6) | Range (mcg) | 38-44 | 2-2 | 31-38 | 8-12 | 89-103 |
| 0° | Mean (mcg) | 43 | 2 | 33 | 9 | 98 |
| (n = 6) | Range (mcg) | 42-45 | 2-2 | 33-34 | 8-10 | 96-99 |
| +45° | Mean (mcg) | 42 | 2 | 33 | 9 | 95 |
| (n = 6) | Range (mcg) | 40-45 | 2-2 | 30-35 | 8-10 | 90-101 |
| +90° | Mean (mcg) | 43 | 2 | 32 | 7 | 93 |
| (n = 6) | Range (mcg) | 41-45 | 2-2 | 29-34 | 6-8 | 88-98 |

AB1002

| | | | | | | |
|---|---|---|---|---|---|---|
| −90° | Mean (mcg) | 49 | 2 | 31 | 10 | 102 |
| (n = 6) | Range (mcg) | 45-54 | 2-2 | 28-34 | 9-11 | 97-111 |
| −45° | Mean (mcg) | 46 | 2 | 29 | 10 | 96 |
| (n = 6) | Range (mcg) | 44-48 | 2-2 | 27-31 | 8-11 | 92-99 |
| 0° | Mean (mcg) | 47 | 2 | 30 | 10 | 99 |
| (n = 6) | Range (mcg) | 46-48 | 2-2 | 28-32 | 8-12 | 95-104 |
| +45° | Mean (mcg) | 46 | 2 | 31 | 10 | 97 |
| (n = 6) | Range (mcg) | 44-47 | 1-2 | 29-33 | 9-11 | 93-102 |
| +90° | Mean (mcg) | 48 | 2 | 32 | 11 | 102 |
| (n = 6) | Range (mcg) | 45-51 | 1-2 | 28-37 | 10-12 | 93-114 |

AB1004

| | | | | | | |
|---|---|---|---|---|---|---|
| −90° | Mean (mcg) | 44 | 2 | 32 | 10 | 97 |
| (n = 6) | Range (mcg) | 40-45 | 1-2 | 31-33 | 10-11 | 94-99 |
| −45° | Mean (mcg) | 44 | 2 | 31 | 10 | 97 |
| (n = 6) | Range (mcg) | 41-46 | 1-2 | 29-35 | 9-12 | 93-100 |
| 0° | Mean (mcg) | 45 | 2 | 33 | 11 | 100 |
| (n = 6) | Range (mcg) | 44-47 | 2-2 | 30-34 | 9-12 | 97-103 |
| +45° | Mean (mcg) | 44 | 2 | 32 | 10 | 97 |
| (n = 6) | Range (mcg) | 43-46 | 1-2 | 29-35 | 8-12 | 92-102 |
| 90° | Mean (mcg) | 45 | 2 | 32 | 10 | 99 |
| (n = 6) | Range (mcg) | 43-47 | 2-2 | 28-36 | 8-12 | 94-104 |

TABLE 9

| Metering | Test | Group 1 | Group 2 | Group 3 | Group 4 | Mass Balance |
|---|---|---|---|---|---|---|
| Orientation | Parameter | (36-58 mcg) | (1-3 mcg) | (22-44 mcg) | (4-17 mcg) | (85-115%) |

AB1001

| | | | | | | |
|---|---|---|---|---|---|---|
| −90° | Mean (mcg) | 43 | 3 | 35 | 9 | 100 |
| (n = 6) | Range (mcg) | 42-44 | 2-4 | 31-42 | 7-11 | 92-113 |
| −45° | Mean (mcg) | 42 | 2 | 33 | 9 | 96 |
| (n = 6) | Range (mcg) | 40-45 | 2-3 | 30-37 | 8-11 | 91-100 |
| 0° | Mean (mcg) | 43 | 2 | 35 | 9 | 99 |
| (n = 6) | Range (mcg) | 40-47 | 2-3 | 30-38 | 8-10 | 92-109 |
| +45° | Mean (mcg) | 42 | 2 | 35 | 10 | 98 |
| (n = 6) | Range (mcg) | 40-44 | 2-2 | 31-38 | 8-12 | 92-104 |
| +90° | Mean (mcg) | 44 | 2 | 34 | 9 | 99 |
| (n = 6) | Range (mcg) | 41-46 | 2-2 | 32-37 | 8-10 | 93-106 |

AB1002

| | | | | | | |
|---|---|---|---|---|---|---|
| −90° | Mean (mcg) | 45 | 2 | 31 | 11 | 98 |
| (n = 6) | Range (mcg) | 42-47 | 2-2 | 29-32 | 10-11 | 91-102 |
| −45° | Mean (mcg) | 46 | 2 | 32 | 11 | 100 |
| (n = 6) | Range (mcg) | 41-50 | 2-2 | 28-42 | 8-15 | 90-115 |
| 0° | Mean (mcg) | 45 | 2 | 31 | 11 | 99 |
| (n = 6) | Range (mcg) | 43-47 | 2-2 | 28-34 | 9-12 | 93-104 |
| +45° | Mean (mcg) | 45 | 2 | 32 | 11 | 99 |
| (n = 6) | Range (mcg) | 43-46 | 2-2 | 29-34 | 9-13 | 94-103 |
| +90° | Mean (mcg) | 44 | 2 | 31 | 11 | 97 |
| (n = 6) | Range (mcg) | 43-45 | 2-2 | 28-33 | 9-12 | 93-101 |

AB1004

| | | | | | | |
|---|---|---|---|---|---|---|
| −90° | Mean (mcg) | 44 | 2 | 32 | 10 | 98 |
| (n = 6) | Range (mcg) | 42-49 | 2-3 | 29-36 | 8-11 | 91-103 |
| −45° | Mean (mcg) | 46 | 2 | 10 | 10 | 101 |
| (n = 6) | Range (mcg) | 43-50 | 2-2 | 29-36 | 9-11 | 95-110 |
| 0° | Mean (mcg) | 44 | 2 | 33 | 11 | 99 |
| (n = 6) | Range (mcg) | 42-48 | 2-2 | 29-37 | 9-12 | 93-104 |
| +45° | Mean (mcg) | 44 | 2 | 35 | 11 | 102 |
| (n = 6) | Range (mcg) | 43-44 | 2-2 | 32-37 | 9-13 | 96-107 |
| +90° | Mean (mcg) | 44 | 2 | 34 | 11 | 101 |
| (n = 6) | Range (mcg) | 41-47 | 1-2 | 30-40 | 8-15 | 95-109 |

All of the results meet the acceptance criteria for the commercialised product specification.

### GENERAL

It should be understood that the foregoing detailed description and preferred embodiments are only illustrative of inhalers constructed in accordance with the present disclosure. Various alternatives and modifications to the presently disclosed inhalers can be devised by those skilled in the art without departing from the spirit and scope of the present disclosure. For example, differing methods of lubrication can be envisaged other than those specifically identified. Accordingly, the present disclosure is intended to embrace all such alternatives and modifications that fall within the spirit and scope of an inhaler as recited in the appended claims.

1. A dry powder inhaler comprising:
a mouthpiece for patient inhalation;
a cover movable about a hinge to open and close the mouthpiece;
a delivery passageway for directing an inhalation induced air flow through the mouthpiece;
a channel extending from the delivery passageway;
a reservoir for containing medicament, the reservoir having a dispensing port connected to the channel;
a cup received in the channel and movable between the dispensing port and the delivery passageway, wherein the cup comprises a cup cam follower;
a cup spring biasing the cup towards the delivery passageway;
a yoke movable between at least a first position and a second position and including a yoke cam;
whereby closing the cover moves the yoke between the first position and the second position such that the yoke cam engages the cup cam follower and urges the cup against the cup spring to the dispensing port;
and wherein the yoke cam, the cup cam follower, or both, is lubricated.

2. A dry powder inhaler according to claim 1, wherein the yoke cam, the cup cam follower, or both are lubricated by application of a surface coating.

3. A dry powder inhaler according to claim 2, wherein the yoke cam, the cup cam follower, or both are lubricated by application of a surface coating of oil.

4. A dry powder inhaler according to claim 3, wherein the oil is a siloxane.

5. A dry powder inhaler according to claim 2, wherein the yoke cam, the cup cam follower, or both are lubricated by application of a surface coating of soap.

6. A dry powder inhaler according to claim 2, wherein the yoke cam, the cup cam follower, or both are lubricated by application of a surface coating of stearic acid.

7. A dry powder inhaler according to claim 1, wherein the yoke cam, the cup cam follower, or both are lubricated by addition of a material additive.

8. A dry powder inhaler according to claim 7, wherein the yoke cam, the cup cam follower, or both comprises a plastics material and the material additive in the plastics is a slip additive.

9. A dry powder inhaler according to claim 8 wherein the plastics material is a polytetrafluoroethylene-containing plastics material.

10. A dry powder inhaler according to claim 8 or 9, wherein the slip additive is a siloxane.

11. A dry powder inhaler according to claim 1, wherein the inhaler additionally comprises at least one cover cam mounted on the mouthpiece cover and movable with the cover between open and closed positions, wherein the cover cam includes at least a first and second cover cam surfaces;
and the yoke includes yoke cam followers which are biased against the cam surfaces by an actuation spring;
wherein the cover cam surfaces are arranged such the yoke cam followers successively engage the first cover cam surface when the cover is closed, and the second cover cam surface when the cover is opened;
and the first cam surface is spaced further from the hinge than the second cam surfaces;
and therefore the yoke cam followers are moved by the actuation spring from the first to the second cover cam surfaces as the cover is opened and thereby the yoke is moved from the second to the first position;
and the yoke cam followers are moved against the actuation spring from the second to the first cover cam surfaces as the cover is closed and thereby the yoke is moved from the first to the second position.

12. A dry powder inhaler according to claim 11, wherein the cover cam includes an additional intermediate cover cam surface between the first and second cover cam surfaces wherein the yoke cam followers are moved by the actuation spring from the first to the intermediate to the second cover cam surface as the cover is opened and the yoke cam followers are moved against the actuation spring from the second to the intermediate to the first cover cam surface as the cover is opened.

13. A dry powder inhaler comprising:
a moving component comprising a plastics material which includes a slip additive; or
wherein the moving component is at least partially coated with an oil, soap or stearic acid.

14. A dry powder inhaler according to claim 13, wherein the oil is a siloxane.

15. A dry powder inhaler according to claim 14, wherein the slip additive is a siloxane.

16. A dry powder inhaler according to claim 9, wherein the slip additive is a siloxane.
